(19) United States
(12) Patent Application Publication
(10) Pub. No.: US 2026/0207270 A1
(43) Pub. Date: Jul. 23, 2026

(54) SURGICAL ROBOT ARM, SURGICAL SYSTEM AND METHOD FOR CONTROLLING A SURGICAL ROBOT ARM

(71) Applicant: KARL STORZ SE & Co. KG, Tuttlingen (DE)

(72) Inventors: Sebastian WAGNER, Tuttlingen (DE); Martin HOFFMANN, Tuttlingen (DE)

(73) Assignee: KARL STORZ SE & Co. KG, Tuttlingen (DE)

(21) Appl. No.: 19/134,185

(22) PCT Filed: Nov. 29, 2023
(86) PCT No.: PCT/EP2023/083602

§ 371 Date: May 29, 2025

(30) Foreign Application Priority Data
Nov. 30, 2022 (DE) 10 2022 131 661.0

(51) Int. Cl. A61B 34/30

(52) U.S. Cl. CPC A61B 34/30; A61B 2034/301; A61B 2034/305

(57) ABSTRACT

Surgical robot arm with a first arm, a second arm and a third arm, wherein a work element having a longitudinal axis is arranged at a second end of the third arm, the first axis, the second axis, the third axis and the longitudinal axis intersect at a common point of rest, and, upon movement of one or more of the first, second or third arm, the point of rest remains stationary. Also disclosed are a surgical system, having a mount and a surgical robot arm, and a method for controlling a surgical robot arm.

### CROSS-REFERENCE TO RELATED APPLICATIONS

This application is the U.S. national stage of PCT/EP2023/083602 filed on Nov. 29, 2023, which claims priority of German Patent Application No. DE 10 2022 131 661.0, filed on Nov. 30, 2022, the contents of which are incorporated herein.

### TECHNICAL FIELD

The disclosure relates to a surgical robot arm, a surgical system with a mount and with a robot arm, and a method for controlling a surgical robot arm.

### BACKGROUND

During laparoscopic surgery, instruments must be inserted through small openings in the patient's body. The point where this occurs should be subjected to as little stress as possible to avoid irritation or even injury.

The required instruments are often inserted using a trocar. The point just mentioned is often referred to as the trocar point. The secure determination of the trocar point is an important factor in the design of a robotic assistance system. This ensures that, after correct initial positioning, the point at which an instrument is inserted into the body is subjected to as little stress as possible, even if the positioning configuration of the robot arm is changed.

Due to the wide range of control options on a robot arm, it is possible to carry out such a low stress change between positioning configurations of the robot arm or robot arms by appropriately controlling the actuators, in particular motors. The definition of a “fixed” point in space without assigning it any useful geometry, in particular the mechanics of the robot arm, is called Remote Center of Motion (RCM).

However, according to the normative definition, the determination of the trocar point by means of software must always be considered unsafe, since errors in the control or errors in the implementation of an otherwise correct control can occur. Such errors can have serious consequences for the patient, so that very complex protective measures must be taken to prevent such errors.

There are various shortcomings and problems with the current solutions. For example, self-collision of arms in robot systems with multiple robot arms is a significant problem when working with one or more trocar points. It can be difficult to switch between positioning configurations of the robot arm or arms while minimizing stress on the trocar points.

For serial robot arms that have at least seven degrees of freedom, switching between positioning configurations of the robot arms can generally be achieved and collisions between the robot arms can generally be avoided. The disadvantage of this, however, is that such a serial robot must always maintain its trocar point using software and must therefore generally be considered unsafe without further measures.

### SUMMARY

A problem addressed by the present disclosure is therefore that of providing an improved robot arm, a corresponding system and a corresponding method for controlling a robot arm, which offer a solution to the above-mentioned problems.

According to a first aspect, the problem is solved by a surgical robot arm with a first arm, which is designed to be arranged on a mount with a first end of the first arm so as to be rotatable about a first axis, a second arm, which is arranged with a first end of the second arm at a second end of the first arm so as to be pivotable about a second axis relative to the first arm, a third arm, which is arranged with a first end of the third arm at a second end of the second arm so as to be pivotable about a third axis relative to the second arm, wherein a work element with a longitudinal axis is arranged at a second end of the third arm, wherein the first axis, the second axis, the third axis and the longitudinal axis intersect at a common point of rest, and wherein, upon movement of one or more of the first, second or third arms, the point of rest remains stationary.

A technical approach of the disclosure is to provide a robot arm as an RCM mechanism that is capable of maintaining the identical orientation of the work element and thus of an instrument guided therein in different position configurations of the robot arm.

A robot arm with three axes of rotation about a point of rest, in particular the mentioned trocar point, is shown. In addition to the three rotation axes of the robot arm, there is a linear axis that runs linearly to the longitudinal axis, i.e., straight to an instrument axis of an instrument guided in the work element.

The robot arm has at least one more degree of freedom around the point of rest or trocar point than is necessary for complete positioning as such. The additional degree of freedom allows different position configurations of the arms of the robot arm, so that repositioning is possible without changing the orientation of the work element or the instrument. This can also expand the possibilities for avoiding a collision or self-collision. Due to the mechanical nature of the robot arm, the potential risks are relatively low and easy to control.

This completely solves the problem.

In a preferred embodiment, the third arm has a first portion with the first end of the third arm and a second portion with the second end of the third arm, wherein the second portion is at an angle to the first portion and extends parallel to the longitudinal axis.

This embodiment allows a compact design of the robot arm.

In a further preferred embodiment, the third arm can be positioned such that the longitudinal axis is parallel to the first axis and in particular coincides with the first axis.

This embodiment allows a compact design of the robot arm.

In a further preferred embodiment, a first angle between the first axis and the second axis is between 20° and 40°, preferably between 25° and 35°, particularly preferably at least approximately 30° and in particular 30°.

This embodiment is considered particularly advantageous for practical use.

In a further preferred embodiment, a second angle between the second axis and the third axis is between 20° and 40°, preferably between 25° and 35°, particularly preferably at least approximately 30° and in particular 30°.

This embodiment is considered particularly advantageous for practical use.

In a further preferred embodiment, a first angle between the first axis and the second axis and a second angle between the second axis and the third axis differ in size by no more than 10°, preferably no more than 5°, and are particularly preferably at least approximately the same size and are in particular the same size.

This embodiment is considered particularly advantageous for practical use.

In a further preferred embodiment, the second arm is designed in the shape of a first circular arc and an imaginary center of a first circle on which the first circular arc lies is the point of rest.

This embodiment is considered particularly advantageous for the movement of the arms of the robot arm relative to each other.

In a further preferred embodiment, a first portion of the third arm is designed in the shape of a second circular arc and an imaginary center of a second circle on which the second circular arc lies is the point of rest.

This embodiment is considered particularly advantageous for the movement of the arms of the robot arm relative to each other.

In a further preferred embodiment, the second arm is spaced from the third arm in the direction of the point of rest.

This design is considered particularly advantageous for the operation of the robot arm.

In a further preferred embodiment, the first arm is guided away from the first axis in a first portion of the first arm and is guided parallel to the first axis and spaced from the first axis in a second portion of the first arm.

This embodiment allows a compact design of the robot arm.

According to a second aspect, the object is achieved by a surgical system having a mount and a previously described surgical robot arm, wherein the robot arm is arranged on the mount and is rotatable about the first axis relative to the mount.

According to a third aspect, the object is achieved by a method for controlling a previously described surgical robot arm, wherein the first, second and third arms are moved from a first position configuration into a second position configuration, wherein the point of rest remains stationary due to the mechanical design of the robot arm.

Further embodiments, as well as some of the advantages associated with these and other embodiments, are made apparent and better understood from the following detailed description with reference to the accompanying figures. Objects or parts thereof which are substantially the same or similar may be provided with the same reference signs. The figures are merely a schematic representation of an embodiment of the invention. Further advantages are evident from the following description of the drawings. The drawings show an exemplary embodiment of the invention. The drawings, the description, and the claims contain numerous features in combination. It is understood that the features mentioned above and those to be explained below can be used not only in the combination specified in each case, but also in other combinations or on their own, without departing from the scope of the present invention.

An exemplary embodiment of the disclosure is shown in the drawing and is explained in more detail in the following description.

### BRIEF DESCRIPTION OF DRAWINGS

FIG. 1 shows an embodiment of a system with a mount and an embodiment of a robot arm.

### DETAILED DESCRIPTION OF PREFERRED EMBODIMENTS

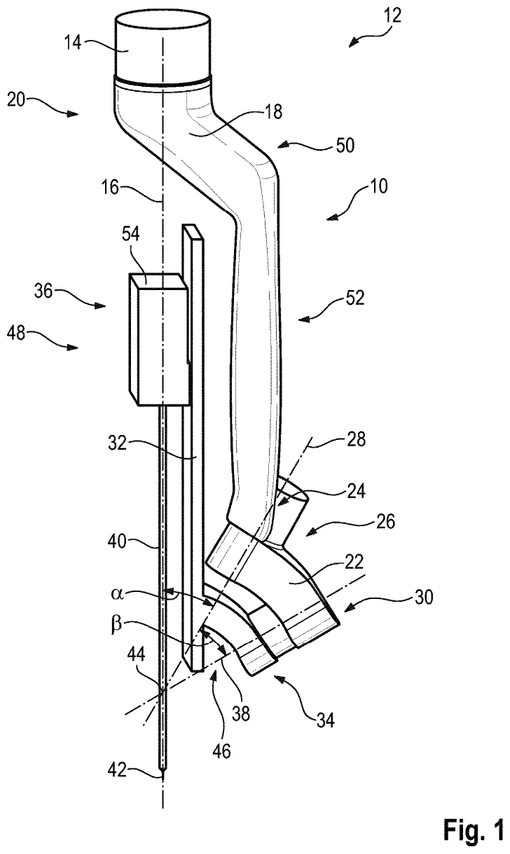

FIG. 1 shows an embodiment of a surgical system 12 with a mount 14 and a surgical robot arm 10, wherein the robot arm 10 is arranged on the mount 14 and is rotatable relative to the mount 14 about a first axis 16. Specifically, a first arm 18 of the robot arm 10 is designed to be arranged on the mount 14 with a first end 20 of the first arm 18 so as to be rotatable about the first axis 16.

The robot arm 10 also has a second arm 22, which is arranged with a first end 26 of the second arm 22 at a second end 24 of the first arm 18 so as to be pivotable about a second axis 28 relative to the first arm 18. The robot arm 10 also has a third arm 32 which is arranged with a first end 34 of the third arm 32 at a second end 30 of the second arm 22 so as to be pivotable about a third axis 38 relative to the second arm 22, At a second end 36 of the third arm 32, there is arranged a work element 40 with a longitudinal axis 42. The first axis 16, the second axis 28, the third axis 38 and the longitudinal axis 42 intersect at a common point of rest 44. If one or more of the first, second or third arms 16, 28, 38 are moved, the point of rest 44 remains stationary.

The third arm 32 has a first portion 46 with the first end 34 of the third arm 32 and a second portion 48 with the second end 36 of the third arm 32, wherein the second portion 48 is at an angle to the first portion 46 and extends parallel to the longitudinal axis 42. The third arm 32 can be positioned so that the longitudinal axis 42 is parallel to the first axis 16 and in particular coincides with the first axis 16, as shown in this exemplary embodiment.

A first angle a between the first axis 16 and the second axis 28 is between 20° and 40°, preferably between 25° and 35°, particularly preferably at least approximately 30° and in particular 30°, as shown in this exemplary embodiment. A second angle ß between second axis 28 and third axis 38 is between 20° and 40°, preferably between 25° and 35°, particularly preferably at least approximately 30° and in particular 30°, as shown in this exemplary embodiment.

The first angle a and the second angle ß differ in size by no more than 10°, preferably no more than 5°, and are particularly preferably at least approximately the same size and are in particular the same size, as shown in this exemplary embodiment.

The second arm 22 is designed in the shape of a first circular arc and an imaginary center of a first circle on which the first circular arc lies is the point of rest 44. The first portion 46 of the third arm 32 is designed in the shape of a second circular arc and an imaginary center of a second circle on which the second circular arc lies is the point of rest 44.

The second arm 22 is spaced from the third arm 32 in the direction of the point of rest 44. In addition, the first arm 18 is guided away from the first axis 16 in a first portion 50 of the first arm 18 and is guided parallel to the first axis 16 and spaced from the first axis 16 in a second portion 52 of the first arm 18.

The work element 40 is designed here as a trocar, so that the point of rest 44 can also be referred to as the trocar point. An attachment 54 at the second end 36 of the first arm 32 holds the work element 40, i.e., the trocar, and provides an insertion aid for inserting a medical instrument into the work element 40.

Further embodiments, as well as some of the advantages associated with these and other embodiments, are made apparent and better understood from the following detailed description with reference to the accompanying figures. Objects or parts thereof which are substantially the same or similar may be provided with the same reference signs. The figures are merely a schematic representation of an embodiment of the invention. Further advantages are evident from the following description of the drawings. The drawings show an exemplary embodiment of the invention. The drawings, the description, and the claims contain numerous features in combination. It is understood that the features mentioned above and those to be explained below can be used not only in the combination specified in each case, but also in other combinations or on their own, without departing from the scope of the present invention.

Surgical robot arm 10 with a first arm 18, a second arm 22 and a third arm 32, wherein a work element 40 having a longitudinal axis 42 is arranged at a second end 36 of the third arm 32, the first axis 16, the second axis 28, the third axis 38 and the longitudinal axis 42 intersect at a common point of rest 44, and, upon movement of one or more of the first, second or third arm 18, 22, 32, the point of rest remains stationary. Further disclosed are a surgical system 12 having a mount 14 and a surgical robot arm 10 and a method for controlling a surgical robot arm 10.

1. A surgical robot arm comprising:
a first arm, which is designed to be arranged on a mount with a first end of the first arm so as to be rotatable about a first axis, a second arm, which is arranged with a first end of the second arm at a second end of the first arm so as to be pivotable about a second axis relative to the first arm,
a third arm, which is arranged with a first end of the third arm at a second end of the second arm so as to be pivotable about a third axis relative to the second arm,
wherein a work element with a longitudinal axis is arranged at a second end of the third arm,
wherein the first axis, the second axis, the third axis and the longitudinal axis intersect at a common point of rest, and
wherein, upon movement of one or more of the first, second or third arms, the point of rest remains stationary.

2. The surgical robot arm according to claim 1, wherein the third arm has a first portion with the first end of the third arm and a second portion with the second end of the third arm, wherein the second portion is at an angle to the first portion and extends parallel to the longitudinal axis.

3. The surgical robot arm according to claim 1, wherein the third arm can be positioned such that the longitudinal axis is parallel to the first axis and in particular coincides with the first axis.

4. The surgical robot arm according to claim 1, wherein a first angle (α) between the first axis and the second axis is between 20° and 40°, preferably between 25° and 35°, particularly preferably at least approximately 30° and in particular 30°.

5. The surgical robot arm according to claim 1, wherein a second angle (β) between the second axis and the third axis is between 20° and 40°, preferably between 25° and 35°, particularly preferably at least approximately 30° and in particular 30°.

6. The surgical robot arm according to claim 1, wherein a first angle between the first axis and the second axis and a second angle between the second axis and the third axis differ in size by no more than 10°, preferably no more than 5°, and are particularly preferably at least approximately the same size and are in particular the same size.

7. The surgical robot arm according to claim 1, wherein the second arm is designed in the shape of a first circular arc and an imaginary center of a first circle on which the first circular arc lies is the point of rest.

8. The surgical robot arm according to claim 1, wherein a first portion of the third arm is designed in the shape of a second circular arc and an imaginary center of a second circle on which the second circular arc lies is the point of rest.

9. The surgical robot arm according to claim 1, wherein the second arm is spaced from the third arm in the direction of the point of rest.

10. The surgical robot arm according to claim 1, wherein the first arm is guided away from the first axis in a first portion of the first arm and is guided parallel to the first axis and spaced from the first axis in a second portion of the first arm.

11. A surgical system comprising a mount and the surgical robot arm according to claim 1, wherein the robot arm is arranged on the mount and is rotatable relative to the mount about the first axis.

12. A method for controlling athe surgical robot arm according to claim 1, wherein the first, second and third arms are moved from a first position configuration to a second position configuration, wherein the point of rest remains stationary due to the mechanical design of the robot arm.
